(19) United States
(12) Patent Application Publication
(10) Pub. No.: US 2017/0276633 A1
(43) Pub. Date: Sep. 28, 2017

(54) DETERMINING GLUCOSE CONTENT OF A SAMPLE

(71) Applicant: Mologic Limited, Thurleigh, Bedfordshire (GB)

(72) Inventors: Andrew John Austin, Great Addington, Northants (GB); Paul James Davis, Sharnbrook, Bedford (US)

(73) Assignee: Mologic Limited, Thurleigh, Bedfordshire (GB)

(21) Appl. No.: 15/512,287

(22) PCT Filed: Sep. 21, 2015
(86) PCT No.: PCT/GB2015/052710

§ 371 Date: Mar. 17, 2017

(30) Foreign Application Priority Data
Sep. 19, 2014 (GB) 1416588.0
Mar. 26, 2015 (GB) 1505198.0

(51) Int. Cl. G01N 27/327; G01N 33/66

(52) U.S. Cl. CPC G01N 27/3277; G01N 33/66

(57) ABSTRACT

Non-enzymatic approaches to measuring glucose are based on the direct oxidation of glucose using unmodified copper metal electrodes. A potential is applied to a copper measurement/working electrode, which potential is monitored by a separate reference electrode and the current within the system is balanced with a counter electrode. The presence of the ionized glucose in the sample can then be determined electrochemically. Disclosed herein are methods, devices, and test systems which utilise this novel approach.

### INTRODUCTION

A number of metals are known to oxidise carbohydrates under alkaline conditions, and this concept has been used in commercial applications, such as for example in flow-through detectors used for monitoring of separation of carbohydrates by HPLC. The literature contains several references that describe detection of carbohydrates, including glucose, using metals such as platinum, gold, silver and copper; often involving complex treatments and preparation to modify the metal surface prior to measurement [Luo et al, Journal of Electroanalytical Chemistry, 1995, v387, pp 87-94, Characterisation of carbohydrate oxidation at copper electrodes; Marioli et al, Electrochim. Acta 1992, v37(7), pp 1187-1197, Electrochemical characterisation of carbohydrate oxidation at copper electrodes; Rahman et al, Sensors, 2010, 10, pp 4855-4886, A Comprehensive Review of Glucose Biosensors Based on Nanostructured Metal-Oxides; Toghill et al, Int. J. Electrochem. Sci., 2010, v5, pp 1246-1301, Electrochemical Non-enzymatic Glucose Sensors: A Perspective and an Evaluation; Sivasankari et al, International Journal of Pharmacy and Biological Sciences, 2012, v2(1), pp 188-195, NON-ENZYMATIC AMPEROMETRIC GLUCOSE BIOSENSOR BASED ON COPPER HEXACYANOFERRATE-FILM MODIFIED-GNP-GRAPHITE COMPOSITE ELECTRODE; the contents of which are incorporated herein]. However to date, there has been no disclosure in the literature or commercial application or exploitation of the use of unmodified copper metal electrode technology in a point of care test for the non-enzymatic measurement of glucose in finger prick blood.

### SUMMARY OF THE INVENTION

Relevant paragraphs:

1. A method for determining the glucose content of a sample comprising causing complete ionisation of the glucose and determining the ionised glucose electrochemically.
2. A method for determining the glucose content of a sample comprising ionising the glucose while the sample is in contact with an un-modified copper electrode and determining the quantity of ionised glucose by detecting changes of current at one or more pre-determined voltage settings.
3. The method of paragraph 1 or 2 where in the conditions causing ionisation of glucose comprises alkalisation of the sample.
4. The method of paragraph 3 wherein the alkalisation comprises increasing the pH of the sample to at least pH14.
5. The method of paragraph 3 wherein the alkalisation is caused by mixing the sample with a strong base.
6. The method of paragraph 5 wherein the strong base is sodium hydroxide, potassium hydroxide, barium hydroxide, ammonium, ammonium hydroxide or methylammonium.
7. The method of any one of paragraphs 1 to 6 wherein the electrochemical detection comprises electro-catalysis
8. The method of paragraph 7 wherein the electro-catalysis comprises oxidation of copper.
9. The method of paragraph 8 wherein the oxidation of copper comprises oxidation of copper 2+ to copper 3+.
10. The method of any one of paragraphs 1 to 9 wherein the determination is by voltammetry.
11. The method of paragraph 9 wherein the voltammetry is sweeping voltammetry.
12. The method of paragraph 9 wherein the voltammetry is cyclic voltammetry.
13. The method of paragraphs 10 or 11 wherein the voltammetry sweeps across a range of 500 to 1200 mV.
14. The method of paragraph 11 or 13 wherein the sweeping voltammetry is forward and/or reverse sweeping.
15. The method of any one of paragraphs 1-14 where in the sample is blood, plasma, serum, urine tears, saliva, or CSF.
16. The method of any one of paragraphs 1 to 15 which further comprises mixing the sample with a polyion.
17. The method of paragraph 16 wherein the polyion is a polyanion.
18. The method of paragraph 16 wherein the polyion is a polycation.
19. The method of paragraph 16 where in the polyion is a polyzwitterion.
20. The method of paragraph 16 wherein the polyion is EDTA and/or, polyethyeleneimine.
21. The method of any one of paragraphs 1 to 20 further comprising mixing the sample with a surfactant.
22. The method of paragraph 21 wherein the surfactant is sorbate.
23. A device for determining the glucose content of a sample comprising a sample analysis area wherein the sample analysis area comprises electrodes and pre-deposited reagent for alkalisation of the sample.
24. The device of paragraph 23 wherein the electrodes comprise metals or conducting polymers.
25. The device of paragraph 23 or 24 wherein the electrodes comprise copper working electrode, a silver/silver chloride reference electrode and a platinum counter electrode.
26. The device of paragraph 23 or 24 wherein the working, counter and reference electrodes are all gold.
27. The device of paragraph 23 or 24 wherein the working and counter electrodes are gold and the reference electrode is silver/silver chloride.
28. The device of paragraph 23 or 24 wherein the electrodes comprise gold working electrode, a silver/silver chloride reference electrode and a platinum counter electrode.
29. The device of paragraph 23 or 24 wherein the working, counter and reference electrodes are all copper.
30. The device of paragraph 23 or 24 wherein the working and counter electrodes are copper and the reference electrode is silver/silver chloride.
31. The device of any one of paragraphs 23 to 30 wherein the copper and platinum electrodes comprise evaporated film electrodes.
32. The device of any one of paragraphs 23 to 31 wherein the reagent for alkalisation of glucose comprises a strong base.
33. The device of paragraph 32 wherein the strong base comprises sodium hydroxide, potassium hydroxide, Barium hydroxide, ammonium, ammonium hydroxide or methylammonium.
34. The device of any one of paragraphs 23 to 33 wherein the reagent for alkalisation of glucose further comprises a polyion.
35. The device of paragraph 34 wherein the polyion comprises EDTA and or polyethyleneimine.
36. The device of any one of paragraphs 23 to 35 wherein the reagent for alkalisation for the sample further comprises a surfactant.
37. The device of any one of paragraphs 23 to 36 wherein the electrodes and reagent for alkalisation of the sample are physically separate but fluidically connected.
38. The device of any one of paragraphs 23 to 37 where the electrodes are capable of electro-catalysis of ionised glucose.
39. The device of paragraph 25 wherein the electrodes comprise alternative electrode arrangements.
40. The device of any one of paragraphs 23 to 29 wherein glucose is determined electrochemically following ionisation and electrocatalysis of glucose.
41. The device of any one of paragraphs 23 to 40 wherein the glucose can be determined at more than one electrode potential.
42. A biosensor, comprising;
a base layer having disposed thereon at least one conductive track extending from a first end to a second end, wherein the conductive track comprises copper;
an assay zone at the first end of the base layer, comprising a reagent capable of increasing the pH of a sample applied to the assay zone;
a terminal at the second end of the base for connection of the at least one conductive track to a processor.
   43. The biosensor of paragraph 42 further comprising a capillary chamber at the first end for receiving a sample of body fluid, wherein the capillary chamber is disposed over the assay zone such that a portion of the at least one conductive track is exposed within the capillary chamber.
   44. The biosensor of paragraph 42 or 43, wherein the base layer has disposed thereon at least three conductive tracks, each conductive track being electrically insulated from the other.
   45. The biosensor of paragraph 44 wherein the at least three conductive tracks comprise copper and wherein a portion of the at least three conductive tracks is exposed within the capillary chamber, and further wherein the capillary chamber contains the pH altering reagent.
   46. The biosensor of any one of paragraphs 43-45 wherein the pH altering reagent is disposed on an inner surface of the capillary chamber.
   47. The biosensor of any one of paragraphs 44-46 wherein the pH altering reagent is disposed on the base layer, but not in contact with the at least three conductive tracks within the capillary chamber.
   48. The biosensor of any one of paragraphs 43-45 wherein the pH altering reagent is disposed within the capillary chamber.
   49. The biosensor of any one of paragraphs 42-48 wherein the at least three conductive tracks define at least one measurement electrode, at least one reference electrode and at least one counter electrode, and wherein the measurement electrode, counter electrode and reference electrode are located within the capillary chamber in the assay zone.
   50. A method, comprising:
   ionizing glucose present in whole blood; and
   electrochemically determining the presence of the ionized glucose in the whole blood.
   51. The method of paragraph 50, wherein ionizing the glucose comprises combining the whole blood with a dried reagent.
   52. The method of paragraph 51, wherein the dried reagent is present in an amount sufficient to increase the pH of the whole blood by an amount sufficient to ionize the glucose.
   53. The method of any one of paragraphs 50-52, wherein the electrochemically determining is performed in a chamber having a total volume of less than about 5 microliters.
   54. The method of any one of paragraphs 50-53, wherein the electrochemically determining comprises electrochemically determining the ionized glucose via an electrochemical circuit comprising at least one copper electrode in contact with the whole blood.
   55. The method of any one of paragraphs 50-54, wherein the method is performed in the absence of enzymes/mediators.
   56. A test strip for determining the presence of glucose, comprising:
   a capillary chamber defining a total volume of less than about 2.5 microliters;
   at least one copper electrode in electrochemical communication with the capillary chamber; and
   a dried reagent present in an amount sufficient to increase a pH of a whole blood sample introduced into the capillary chamber and filling the volume of the capillary chamber by an amount sufficient to ionize glucose present in the whole blood.
   57. The device of paragraph 56 wherein the test strip comprises three copper electrodes configured as:
i) a working electrode at which measurement of glucose oxidation occurs;
ii) a counter electrode, which supplies or consumes electrons in response to the reaction at the working electrode; and
iii) a reference electrode, which acts to monitor and maintain the potential applied between the working electrode and counter electrode.
   58. The device of paragraphs 56 or 57 wherein the capillary chamber defines a volume of less than about 2 microlitres.
   59. The device of paragraphs 56 or 57 wherein the capillary chamber defines a volume of less than about 1 microlitre.
   60. The device of paragraphs 56 or 57 wherein the capillary chamber defines a volume of less than about 0.5 microlitres.
   61. The device of any one of paragraphs 56 to 60 wherein the dried reagent is disposed on a surface of the capillary chamber not in direct contact with the one or more copper electrodes.
   62. The device of any one of paragraphs 56 to 61 wherein the dried reagent comprises base and a surfactant.
   63. The device of paragraph 62 wherein the surfactant is polyvinyl alcohol and the base is sodium hydroxide.
   64. A method of determining the quantity of glucose in a sample of blood obtained from a finger prick or alternate site using a device of paragraphs 56 to 63, comprising;
removing the test strip from a storage compartment;
inserting the test strip into a meter and following the instructions presented on the display of the meter;
pricking a finger or alternate site to release a drop of blood;
contacting the drop of blood with the sample port on the test strip;
removing the test strip from the drop of blood when the meter indicates sufficient sample has been acquired on the test strip;
allowing the blood to react in the test strip for at least 1 second; and
displaying a blood glucose concentration on the display of the meter.
   65. The method of paragraph 64, wherein the blood reacts in the test strip for at least 3 seconds before a glucose concentration is displayed.
   66. The method of paragraph 64, wherein the blood reacts in the test strip for at least 5 seconds before a glucose concentration is displayed.
   67. The method of paragraph 64, wherein the blood reacts in the test strip for at least 7 seconds before a glucose concentration is displayed.
   68. The method of paragraph 64, wherein the blood reacts in the test strip for at least 10 second before a glucose concentration is displayed.
   69. The method of any one of paragraphs 64 to 68 wherein no more than 2.5 microlitres of blood has been acquired on the test strip.
   70. The method of any one of paragraphs 64 to 68 wherein no more than 1.5 microlitres of blood has been acquired on the test strip.
   71. The method of any one of paragraph 64 to 68 wherein no more than 1 microlitre of blood has been acquired on the test strip.
   72. The method of any one of paragraphs 64 to 68 wherein no more than 0.5 microlitres of blood has been acquired on the test strip.

### DESCRIPTION OF THE FIGURES

FIG. 1 shows an example of a general 3-electrode design according to the invention.

FIG. 2 shows an expanded area of FIG. 1 showing the electrode design which will be exposed to the sample for testing.

FIG. 3: diagram to show the position of the block mask to leave an enlarged exposed electrode area.

FIG. 4: diagram to show the position of a typical capillary chamber located over the 3-electrode design.

FIG. 5: current response for low range of glucose in whole sheep blood using 3× copper electrodes (WE,CE,RE).

FIG. 6: current response for high range of glucose in 0.5M NaOH using 3× copper electrodes (WE,CE,RE).

FIG. 7: current response from fast chrono method showing the high range glucose response.

FIG. 8: Mean ACuTEGA signals in whole sheep blood, spiked with various glucose concentrations, showing SD and CofV for each value (n=5 for each point).

FIG. 9: Current/time curves of repeat ACuTEGA glucose assays in glucose-spiked sheep blood to show speed of response and precision (repeatability).

FIG. 10: Mean ACuTEGA signals in whole sheep blood, spiked with glucose at 1, 3 and 5 mM to prove adequate performance of the system in the clinically important range (n=5 for each point).

FIG. 11: Comparative ACuTEGA signal responses from glucose and maltose under identical conditions. Note that 15 mM maltose gives the same signal as 1 mM glucose.

FIG. 12: Dose response profiles of the ACuTEGA system across the most clinically relevant range of 0-10 mM

FIG. 13: Dose response profiles of the ACuTEGA system up to 30 mM

### DETAILED DESCRIPTION

A new non-enzymatic approach to measuring glucose has been developed and is disclosed herein. The non-enzymatic measurement of glucose is based on the direct oxidation of glucose using unmodified copper metal electrodes. A potential is applied to a copper measurement/working electrode, which potential is monitored by a separate reference electrode and the current within the system is balanced with a counter electrode. The presence of the ionized glucose in the sample can then be determined electrochemically. Disclosed herein are methods, devices, and test systems using this novel approach.

Several exemplary embodiments of copper based measurement systems are described in Table 1. In a first aspect a copper working electrode is used in combination with a silver/silver chloride reference electrode and a platinum counter electrode. In a second embodiment, a copper working electrode is used in combination with a silver/silver chloride counter/reference electrode. In a third aspect a copper working electrode is used in combination with a copper counter/reference electrode. And, in a fourth aspect a copper working electrode is used in combination with a copper reference electrode and a copper counter electrode.

TABLE 1
Copper-based measurement systems

| | Working | Reference | Counter |
|---|---|---|---|
| Test | electrode (WE | electrode (RE) | electrode (CE) |
| Format: | material | material | material |
| 1 | Copper | Ag/AgCl | Platinum |

| | | |
|---|---|---|
| 2 | Copper | Single Ag/AgCl combined RE & |
| | | CE |
| 3 | Copper | Single copper combined RE & |
| | | CE |

| | | | |
|---|---|---|---|
| 4 | Copper | Copper | Copper |

An exemplary copper-based measurement system is based on the All Copper Triple Electrode Glucose Assay (ACuTEGA) technology. Without wishing to be bound by any theory, ACuTEGA may work by directly oxidising glucose which has been converted into an anionic state at a pH sufficient to ionize the glucose. For example, at a pH of about 13 to 14, glucose is subject to electrocatalytic oxidation, peaking at a potential around 900 mV (vs copper reference), yielding 6 formate molecules and 12 electrons for each glucose molecule oxidised. Such an oxidation process yields three or six times the number of electrons per glucose molecule oxidised when compared with more traditional enzyme based self-monitoring blood glucose sensors. Consequently it is expected the measurement of glucose using an ACuTEGA device may allow for more sensitive determination of glucose at lower concentration than might be achieved using more traditional measurement modalities, leading to improved measurement performance.

Under conditions sufficient to ionize glucose in a sample using the novel approach described herein, electrochemical determination of the ionized glucose is not impaired by factors known to interfere with traditional glucose measurements. For example, at pH values in the order of 13 to 14 there is no apparent response detected on the copper electrode from species such as ascorbate, paracetamol, urate, dopamine, etc., which are known to interfere with measurement of glucose at pH close to neutral. Furthermore measurements made using copper electrodes at pH in the region of 14 appears to be unaffected by the haematocrit of the blood under test; which is another factor known to compromise measurement of glucose in traditional enzymic sensor devices. An apparent increase in viscosity of blood that occurs when the pH of the sample is raised to at least 14, appears to cause the blood to be held tightly in the reaction chamber of the test strip. This apparent increase in viscosity appears to negate any effect that haematocrit may otherwise have on the resultant signal measured by the electrode during the oxidation of glucose to formate.

In one aspect a method for determining the glucose content of a sample comprising causing complete ionisation of the glucose and determining the ionised glucose electrochemically, is described. The glucose content of the sample is typically determined by completely ionising the glucose in the sample while it is in contact with an un-modified copper electrode; the quantity of ionised glucose is determined by detecting changes of current at one or more pre-determined voltage settings. The conditions causing ionisation of glucose typically involve alkalisation of the sample; and the pH of the sample is often increased to at least 13 or 14 through the mixing of a strong base, such as for example sodium hydroxide, potassium hydroxide, calcium hydroxide, magnesium hydroxide, barium hydroxide, ammonium, ammonium hydroxide or methylammonium.

The electrochemical detection of glucose oxidation in alkaline solution may be achieved using cyclic voltammetry, chronoamperometry or like techniques which monitor the flow of current when a potential is applied to a working or measurement electrode at which oxidation of the glucose occurs. In one aspect the oxidation of glucose on a copper electrode may follow a process where the copper is changed from copper 2+ to copper 3+. Typically an applied potential in the range of +500 to +1200 mV may be used, depending on the reference electrode being utilised. For example a silver/silver chloride reference electrode may require a different potential be applied compared with using a copper reference electrode.

The strong alkali may be formulated additional additives that may aid drying and resuspension of the dry reagent upon sample addition; such agents may include a polyion, such as a polyanion, a polycation, or a polyzwitterion. In some formulations the polyion may be either EDTA and/or, polyethyeleneimine. The formulation may further include a surfactant, such as for example sorbate, polyvinyl alcohol, saponin.

In another aspect a device for determining the glucose content of a sample that includes a sample analysis area, which includes one or more electrodes and pre-deposited dried reagent for alkalisation of the sample is disclosed. The electrodes may be formed using metals or conducting polymers, including for example, platinum, gold, silver, copper, zinc, ruthenium, palladium, poly(3,4-ethylenedioxythiophene), polypyrrole, polyaniline, polythiophene. In some embodiments the electrodes may include a copper working electrode, a silver/silver chloride reference electrode and a platinum counter electrode; or the working, counter and reference electrodes may all be formed from gold. In other embodiments the working and counter electrodes may be formed from gold and the reference electrode may be of silver/silver chloride; or the electrodes may include a gold working electrode, a silver/silver chloride reference electrode and a platinum counter electrode. In an exemplary embodiment the working, counter and reference electrodes are all formed from copper; or the working and counter electrodes may be formed from copper and the reference electrode from silver/silver chloride. In some embodiments the electrodes and reagent used for alkalisation of the sample are physically separate but fluidically connected; in other cases the reagents are deposited directly over the electrodes. In general, the materials from which the electrodes are made will be capable of direct measurement of any ionised glucose in the sample, leading to a signal that is proportional to a concentration of glucose present.

In an exemplary embodiment, disclosed is a device for the quantitative determination of blood glucose in a sample. For example, the device can be used for determination of glucose in a sample of whole blood. The device may also be used to determine the presence of glucose in plasma, serum, urine and other fluid samples. Whole blood can be readily obtained from a finger prick or other alternate site that is readily accessible, using a lancing device available for personal use. Blood may also be obtained by a suitably qualified phlebotomist using venipuncture. The device utilizes copper electrodes to determine glucose within the sample with no requirement for enzymes or mediator compounds. The device may be a test strip including a capillary chamber, at least one copper electrode, and a dried reagent. In some embodiments, the capillary chamber is in electrochemical communication with the at least one copper electrode. In some embodiments, the dried reagent is present in the capillary chamber. The dried reagent may be present in an amount sufficient to increase the pH of the sample, for example whole blood sample, introduced into the capillary chamber to at least 13 and more preferably to at least 14. The capillary chamber may define a total volume of less than 5 ul, less than 4 ul, less than 3 ul, less than 2.5 ul, less than 1.5 ul, less than 1 ul, less than 0.5 ul.

A device such as a test strip can be stored individually or as a package of strips. A test strip can be used with a meter. For example a test strip can be removed from its packaging or storage compartment and then inserted into a meter. A user would typically use a test strip to determine the quantity of glucose in a sample of blood obtained from a finger prick. The user would first remove the test strip from a storage compartment, which may be an individual foil pouch or similar containment means designed to keep the strip “dry”, or which may be a vial that holds several test strips, which contains a desiccant material to maintain the strips in a “dry” atmosphere. Once removed from the protective container, the user would insert the test strip into a meter and following the instructions presented on the display of the meter. Such instructions will typically indicate the following: prick a finger or alternate site to release a drop of blood; discard the first one or two droplets of blood; contact the drop of blood with the sample port on the test strip; remove the test strip from the drop of blood when the meter indicates sufficient sample has been acquired; wait for the blood to react within the test strip; read the glucose concentration on the display of the meter. The time taken for the blood sample to react within the test strip before the meter displays a glucose reading to the user is typically less than 10 seconds, and more often less than 7 seconds, generally less than 5 seconds and may even be less than 3 seconds and may even be less than 1 second. The technology is thus well suited to providing rapid measurement results, which may be critical in certain circumstances.

Also disclosed herein are biosensors comprising a base layer, an assay zone, and a terminal. The biosensor, can include a base layer having disposed thereon at least one conductive track which extends from one end to the other end of the base layer. The conductive track may be formed using copper. The biosensor also includes an assay zone at one end of the base layer, which may include a dried reagent that is capable of increasing the pH of a sample applied to the assay zone. A terminal at the other end of the base layer is used for making a connection of the at least one conductive track to a microprocessor in an analysis device or meter with which the biosensor is intended to be used. Typically the biosensor will have a capillary chamber at the one end for receiving a sample of body fluid; the capillary chamber is frequently located over the assay zone such that a portion of the at least one conductive track is exposed within the capillary chamber. Therefore when a sample is applied to the biosensor, the sample will be collected within the capillary chamber, where it will make contact with the conductive track. In some cases the biosensor can have at least three conductive tracks one the base layer, with each of the conductive tracks being electrically insulated from the other. In a particular embodiment the biosensor includes at least three conductive tracks that are formed using copper metal, with at least a portion of the three separate conductive tracks being exposed within the capillary chamber and thus accessible for direct contact with a sample applied to the biosensor. Frequently the capillary chamber will include a dried reagent that can alter the pH of a sample applied to the biosensor. The pH altering reagent is typically dried on an inner surface of the capillary chamber; however the pH altering reagent can also be dried down on the base layer, but not in direct contact with the at least three conductive tracks within the capillary chamber. The conductive tracks will generally represent at least one working or measurement electrode, at least one reference electrode and at least one counter electrode, and each of these will exist within the confines of the capillary chamber in the assay zone.

The disclosure further defines a method of measuring glucose that might be present in a sample of whole blood. The method, generally involves completely ionizing any glucose that may be present in a sample of whole blood and then electrochemically determining the presence of the ionized glucose in the whole blood. The process of ionizing the glucose includes combining the whole blood with a dried reagent, which dried reagent is present in an amount sufficient to increase the pH of the whole blood by an amount sufficient to ionize the glucose. The process of electrochemically determining the quantity of ionised glucose is performed in a chamber having a total volume of less than about 5 microliters, more often than not the chamber has a volume of less than 2.5 ul, and in many cases a volume less than 1 ul. The electrochemical determination of the ionized glucose can be achieved using an electrochemical circuit that includes at least one copper electrode which will be in contact with the whole blood. One aspect of the disclosed method is that it does not require the presence of either enzymes or mediators that are utilised in many commercial systems for self-monitoring blood glucose.

The disclosure also includes description of a test strip for determining the presence of glucose in a fluid sample obtained from a human subject. The test strip includes a capillary chamber which defines a total volume of typically less than about 2.5 microliters, and more frequently less than 1 microliter and in some cases less than 0.5 microliters. The test strip also includes at least one copper electrode in electrochemical communication with the capillary chamber; along with a dried reagent present in an amount sufficient to increase a pH of a whole blood sample introduced into the capillary chamber and filling the volume of the capillary chamber by an amount sufficient to ionize glucose present in the whole blood. The test strip will often include at least three copper electrodes that are arranged as: i) a working electrode at which measurement of glucose oxidation occurs; ii) a counter electrode, which supplies or consumes electrons in response to the reaction at the working electrode; and iii) a reference electrode, which acts to monitor and maintain the potential applied between the working electrode and counter electrode. The dried reagent is generally present on a surface of the capillary chamber not in direct contact with the one or more copper electrodes, and it may contain an alkali or base and a surfactant. The base can include sodium hydroxide, potassium hydroxide, calcium hydroxide, magnesium hydroxide, barium hydroxide, ammonium, ammonium hydroxide or methylammonium, and the surfactant can include sorbate, polyvinyl alcohol, or saponin.

### EXAMPLES

### Test Method:

Two different electrochemical tests, cyclic voltammetry (CV) and chronoamperometry (Chrono) were used to characterise the performance of copper working electrodes for direct measurement of glucose under alkali conditions. CV conducts a 3V potential sweep while Chrono applies a single, fixed potential. Both methods have given good detection of glucose in both buffer and blood environments.

### Electrode Preparation:

Copper coated polyester was supplied from Vacuum Depositing Inc. (VDI LLC (Louisville, Ky., USA)). A polyester (polyethylene terephthalate (PET)) sheet was used (Lumirror T62, 750 gauge nominal (˜190 microns)) as the base layer. A tie layer of Chromium and Nickel was sputter coated to act as a bonding layer to improve the adherence of the copper layer to the PET. Following this, copper was sputter coated onto the Cr/Ni tie layer. The tie layer was approximately 3-5 nm in thick, the copper layer was used with a maximum thickness of about 40 nm. No treatment or modification of the pure copper metal surface was performed. The stock copper metal coated polyester supplied by VDI LLC was delivered as a real of material, from which devices for testing were prepared.

In an exemplary embodiment, test sensors were prepared by first removing a section of material approximately 16 cm×16 cm from the master real, being careful not to contaminate the surface. Articles were ultimately cut into strips approximately 5 mm wide by about 35 mm long. The strips of copper coated polyester were pattered using laser etching to define two or more individual electrically insulated tracks; one end of which was used to make electrical connection with a potentiostat or meter that supplied the required voltage polarisation to perform CV or Chrono, as well as acquiring the resultant current corresponding to the oxidation of glucose.

Three separate electrodes (WE, RE and CE) were defined by laser etching, using a Ulyxe laser etching system (Datalogic Automation (supplied by Laserlines Ltd (UK)) was used. The Ulyxe has a 6 w YAG laser, operated at a wavelength of 1064 nm which was demonstrated to cleanly remove both the copper and Cr/Ni tie layer from the PET backing, thus revealing the PET in regions exposed to laser energy. The laser system was typically operated used with the following settings: power (80%), frequency (20,000 Hz), scan speed (500 mm/s), dot delay (5 μs), shot time (1.5 μs), with only a single pass. The lens used was an F254. The Ulyxe was used in-conjunction with a filter extraction system, which removes the vapour debris emitted by the ablation steps.

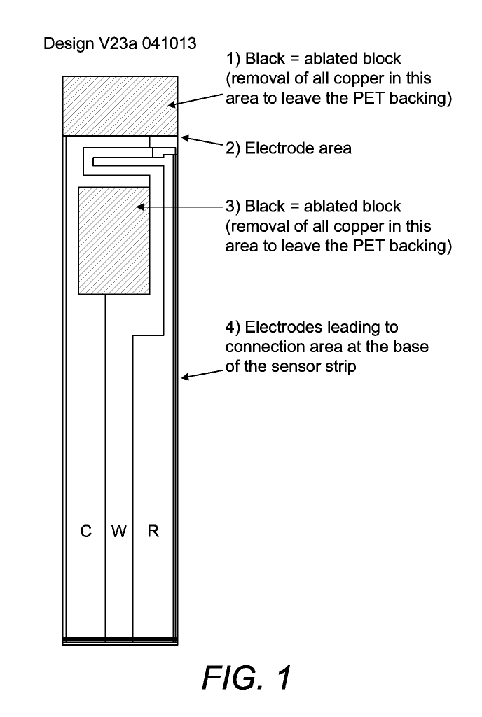

Several designs of electrodes were investigated, each with slight variation in the area of copper metal exposed for each electrode surface. An exemplary design is shown in FIG. 1.

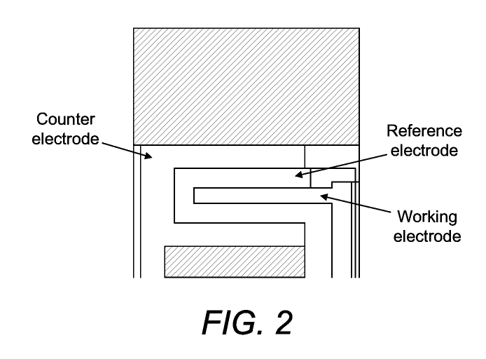

The configuration of the individual electrodes is shown in more detail in FIG. 2. The RE is positioned at the centre of the array, which is in turn surrounded by the WE which itself is surrounded by the CE.

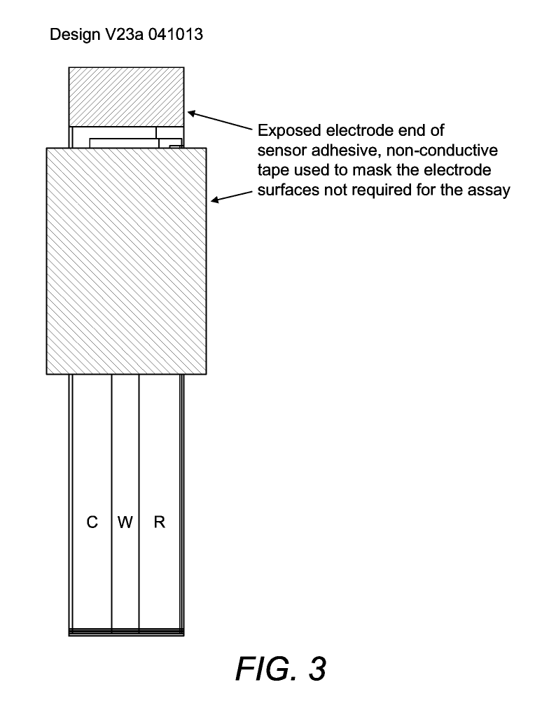

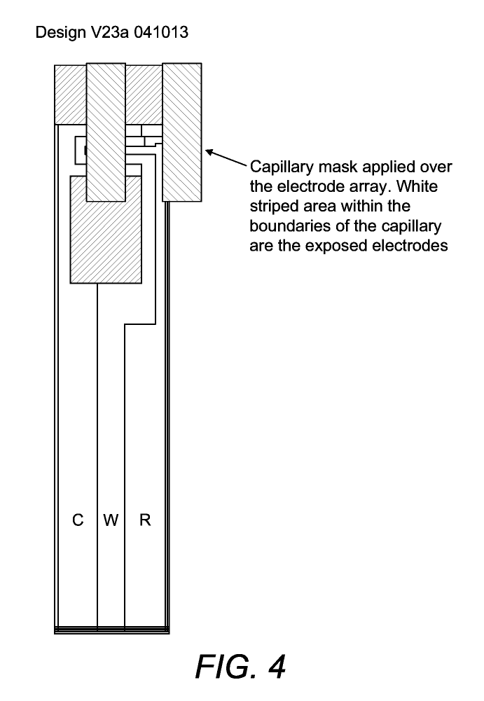

Depending on how the electrode was used, different masking was applied. Under some circumstances a capillary chamber having a volume of no more than 2.5 microlitres was adhered directly over the electrodes. On other occasions a capillary chamber having a volume of no more than 1 microlitre was applied over the electrodes. In general, the end of the electrode is masked off with the use of a non-conductive adhesive tape, or a dielectric insulating ink. FIGS. 3 and 4 depict different approaches to masking off portion of the copper metal as a way of controlling the surface area of metal that may be contacted by a sample.

Once a series of electrodes have been defined on the PET substrate, they were masked with insulating material as shown in FIGS. 3 and 4, and cut from the master sheet to give a sensor with typical dimensions of 35×5.5 mm.

### Hardware:

The following equipment was used.
Potentiostat:
Supplied by Whistonbrook Technologies. Product name is Ezescan. The model typically used is the Ezescan 4. It is a single test potentiostat, with inputs for WE, RE and CE. Software is supplied with the instrument, which allows CV and Chrono methods to be performed. A user interface allows parameters to be determined by the user.
Sensor connection:
A 9-pin D-sub type connector was used for connection to the Ezescan 4 potentiostat. 7-strand copper core wire (conductor area=0.22 mm²) was used for all wiring. A pcb vertical slide connection socket, with 1.27 mm pitch between the pins was used for connection to the copper electrode.

### Materials:

Sodium hydroxide: any high quality, low impurity grade can be used. For example, Sigma-Aldrich Code S5881, >98% purity.

Potassium hydroxide: any high quality, low impurity grade can be used. For example, Sigma-Aldrich Code 484016, >90% purity

Analytical water: <15 MOhm.

Glucose: any high quality, low impurity grade can be used. For example, Sigma-Aldrich Code G8270, >99.5% purity.

General purpose microtitre plate (or any equivalent small volume container).

### Measurement Method for Glucose in Buffer:

The following procedure was performed when measuring glucose in aqueous buffer samples. The example describes testing with a masked electrode as shown in FIG. 3.

1. Individual electrodes are prepared as described under the electrode preparation section.
2. Hydroxide solution is prepared by dissolving pellets in analytical water to give 4M concentration. Preferred cation is potassium, although sodium may also be used.
3. Glucose solution is prepared by dissolving powder in analytical water to give 1M concentration.
4. To an individual microtitre plate well, volumes are dispensed to give a final volume of 200 μl. This volume is sufficient to cover the exposed area of the electrodes when it is submerged to the masked area. The volume is not critical, but there should be sufficient to cover the exposed electrodes.
a. Add hydroxide solution to give the required concentration, for example 0.5M. For example, 25 μl of 4M stock solution in 200 μl final volume.
b. Add glucose solution to the well to give the required concentration, for example, 12 μl of 1M stock in 200 μl final volume to give 30 mM final concentration. Further volumes of glucose are added to wells to give differing glucose concentrations.
c. Make the volume up to 200 μl with analytical water. Aspirate the well to ensure all solutions are mixed well.
5. Take the connection lead, and plug into the potentiostat.
6. Take a single, masked electrode and slide into the connector block, ensuring the electrodes are lined up correctly with the connector pins.
7. Using the user interface with the potentiostat software, choose the method to be used for the test, for example, cyclic voltammetry. Ensure the settings are correct, for example the following settings are typically used:
a. Potential sweep range: −1500 mV forward sweep to +1500 mV with reverse sweep back to −1500 mV.
b. Step interval=10 ms
c. Potential step=10 mV
d. Scan rate equivalent to 1 v/s.
8. Dip the end of the electrode into the test solution, ensuring the exposed area of the sensor is submerged in the test solution. Only submerge the electrodes when the test is ready to be performed. Ensure no air bubbles are trapped or attached to the surface of the electrode.
9. Start the scan, holding the electrode as still as possible to prevent movement of the test sample across the surface of the electrode. The aim is to conduct the test under static conditions.
10. After the scan has been completed, remove the electrode from the test solution and connector and discard.
11. Save the data file.
12. The data is typically imported into a graphics package such as Microsoft Excel. The data is plotted as potential (mv, x-axis) vs current (μA, y-axis). Multiple graphs may be plotted to examine trends throughout the sweep profiles. In addition, specific data (current) can be extracted from the data set which relate to specific peaks which correspond to responses from changes in the presence of glucose.

### Measurement Method for Glucose in Whole Blood:

If blood is to be tested, the analytical water used as described above is replaced with 200 μl whole blood. Typically the blood is collected into citrate-only tubes. Sodium citrate is used as the anti-coagulant, with a final concentration of approximately 0.3%. The whole blood is stored cooled at 4-8° C., until used. If a zero glucose baseline is required, the blood is placed in a 37° C. incubator and monitored with a commercial glucose detection device until the reading is too low to read (typically <1 mM glucose). Glucose may then be spiked back into the depleted blood to give known concentrations of soluble glucose. Differences in the volume of glucose added to the blood sample are compensated for by additional water.

The following procedure is performed when measuring glucose in whole blood samples. The example describes testing with a masked electrode as shown in FIG. 3.

1. Individual electrodes are prepared as described under the electrode preparation section.
2. Hydroxide solution is prepared by dissolving pellets in analytical water to give 4M concentration. Preferred cation is potassium, although sodium may also be used.
3. Glucose solution is prepared by dissolving powder in analytical water to give 1M concentration.
4. To an individual microtitre plate well, volumes are dispensed to give a final volume of 200 μl. This volume is sufficient to cover the exposed area of the electrodes when it is submerged to the masked area. The volume is not critical, but there should be sufficient to cover the exposed electrodes.
a. Add the blood sample to the well.
b. Add glucose solution to the well to give the desired concentration, for example, 12 μl of 1M stock in 200 μl final volume to give 30 mM final concentration. Further volumes of glucose are added to wells to give differing glucose concentrations.
c. Aspirate the well to ensure all solutions are mixed well.
5. Take the connection lead, and plug into the potentiostat.
6. Take a single, masked electrode and slide into the connector block, ensuring the electrodes are lined up correctly with the connector pins.
7. Using the user interface with the potentiostat software, choose the method to be used for the test, for example, cyclic voltammetry. Ensure the settings are correct, for example the following settings are typically used:
a. Potential sweep range: −1500 mV forward sweep to +1500 mV with reverse sweep back to −1500 mV.
b. Step interval=10 ms
c. Potential step=10 mV
d. Scan rate equivalent to 1 v/s.
8. Just prior to testing, add hydroxide solution to the blood to give the desired concentration, for example 0.5M. To achieve this, add 25 μl of 4M stock solution in 200 μl final volume. Mix quickly, because the effect of the sharp rise in pH in the blood is that the blood becomes very viscous and gelatinous.
9. Dip the end of the electrode into the test solution, ensuring the exposed area of the sensor is submerged in the test solution. Only submerge the electrodes when the test is ready to be performed. Ensure no air bubbles are trapped or attached to the surface of the electrode.
10. Start the scan, holding the electrode as still as possible to prevent movement of the test sample across the surface of the electrode. The aim is to conduct the test under static conditions.
11. After the scan has been completed, remove the electrode from the test solution and connector and discard.
12. Save the data file.
13. The data is typically imported into a graphics package such as Microsoft Excel. The data is plotted as potential (mv, x-axis) vs current (μA, y-axis). Multiple graphs may be plotted to examine trends throughout the sweep profiles. In addition, specific data (current) can be extracted from the data set which relate to specific peaks which correspond to responses from changes in the presence of glucose.

### Chronoamperommetry Measurement of Glucose:

A fast chrono method may be used for fixed potential interrogation of the sample. Typically this fixed applied potential is +900 mV, although this should be optimised to reflect the format of the electrode array.

The basic method of sample preparation is the same as described for the cyclic voltammetry methods.

The method used is Fast Chrono with the following parameters:
Potential: +900 mV
Step 10 ms
Time to complete the test: 5 seconds.

### Typical Responses:

### Cyclic Voltammetry Data:

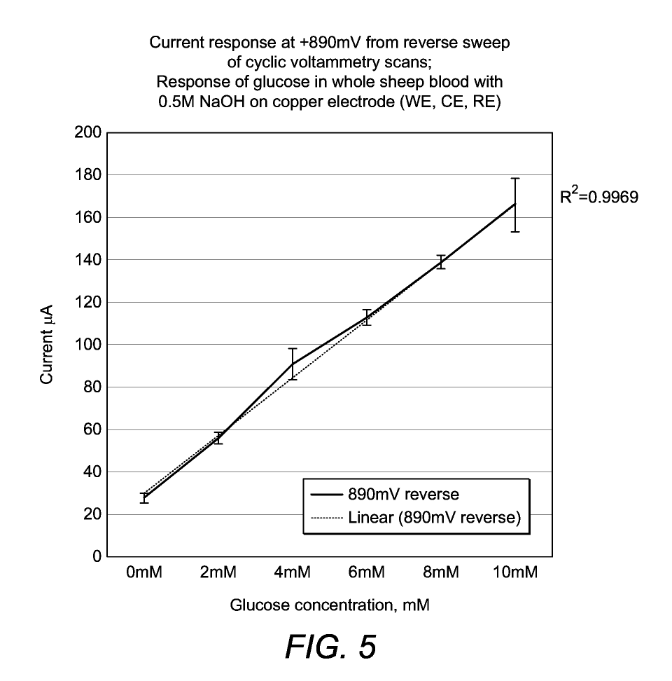

FIG. 5 shows an example of the glucose response, using a laser ablated electrode array, in the presence of whole sheep blood in 0.5M NaOH. The range tested was 0-10 mM to demonstrate the differentiation that was possible with this format.

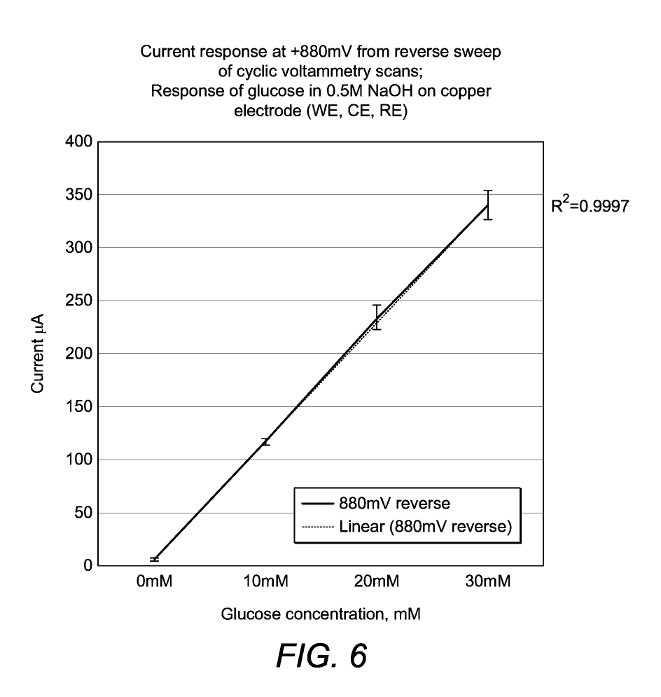

FIG. 6 shows an example of the glucose response using a laser ablated electrode array, in 0.5M NaOH only. The range tested was 0-30 mM to demonstrate the high range linearity of the format.

### Chronoamperometry Data:

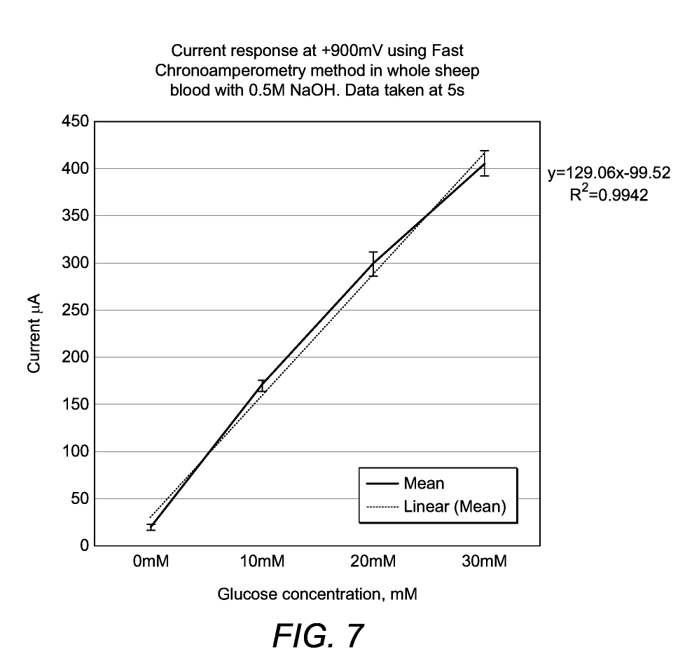

FIG. 7: fast chrono method was used with an applied potential of +900 mV. In this example, individual electrode strips were used rather than a laser ablated array. The result demonstrates the linearity of the glucose response using the chrono single potential method.

The graphs above demonstrate a typical response to the addition of glucose to both just the 0.5M NaOH and to whole sheep blood with 0.5M NaOH.

### ACuTEGA in General Operation:

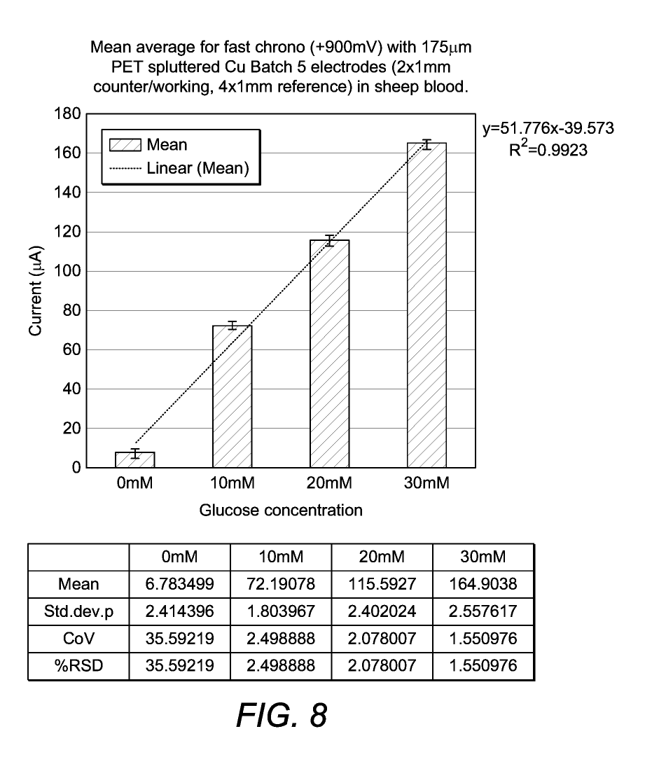

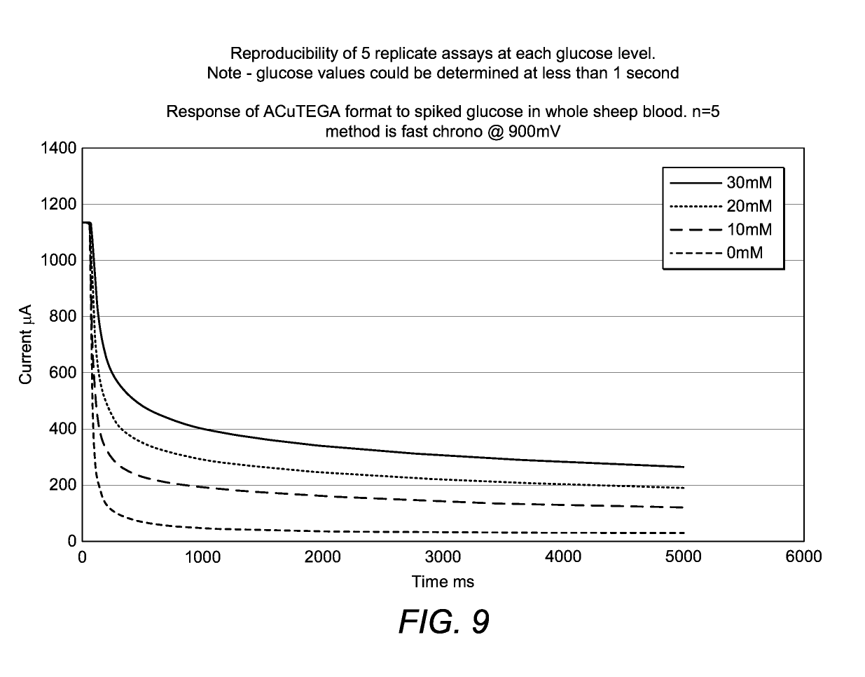

For general testing of the devices depicted in FIG. 3, the fast chrono mode is used, with the potential poised at around +900 mV vs copper reference. A strip is connected to a reader using a push fit connector, after which typically less than 1 μL of finger-stick blood is applied to the end of the strip. As the blood flows into the capillary chamber, it meets and rehydrates the dried sodium hydroxide en-route to the electrode array. An exemplary design of the electrode array as shown in FIGS. 3 and 4, was used. Rehydration of the hydroxide reagent is near instantaneous, causing rapid ionisation of glucose, which typically permit a glucose measurement in less than 5 seconds, frequently less than 3 seconds, and regularly requires less than 1 second from the time of sample introduction to determine a glucose concentration within the sample. The data shown in FIG. 8 represent a dose response curve when glucose was spiked into glucose depleted sheep blood. The chrono time-course profiles for each measurement signal was captured over 5 seconds. The time/current curves are shown in FIG. 9, which clearly show both the rapid response and the reproducibility of the signal in ACuTEGA. In particular it can be seen that stable responses are achieved after just 1 second; allowing determination of the glucose content of the sample to be determined at such time point.

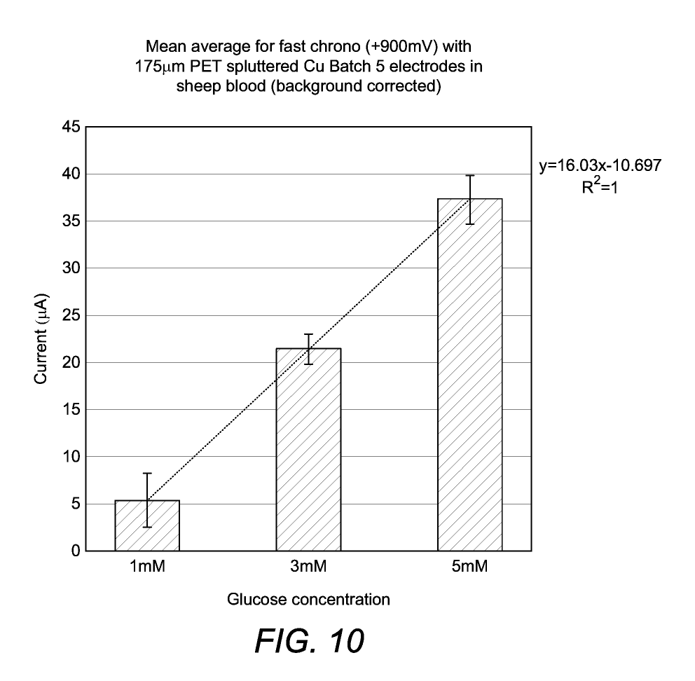

For routine glucose testing by diabetic subjects, it will be essential to gain good discrimination and linearity at glucose levels below 10 mM and ideally below 5 mM—the recommended target level for blood glucose; in this context a series of blood samples spiked with 1 mM, 3 mM and 5 mM glucose were prepared and assayed. The data are shown in FIG. 10.

The ACuTEGA system has been shown to be unaffected by interference from the usual interfering substances that cause problems for enzyme driven tests (paracetamol, ascorbate and urea etc., data not shown), but market forces now requires that glucose tests should discriminate between glucose and maltose. Maltose is a 1,6-linked glucose dimer, and it can sometimes be found in patients who are receiving peritoneal dialysis (who are given intra-peritoneal maltodextrin solutions as “osmotic agents”, known as “Icodextrin”) and very ill cancer patients (who receive oncology medication in which maltodextrin is present as an excipient). There have been rare but high-profile cases in which PQQ-glucose dehydrogenase based enzyme sensors have given falsely elevated readings for glucose, leading to excessive insulin dosing. This is due to the lack of specificity of PQQ-GDH, which will utilise maltose as a substrate in place of glucose. It is reported that maltose levels as high as 3 mM can be found. To the best of our knowledge, higher maltose levels are not encountered.

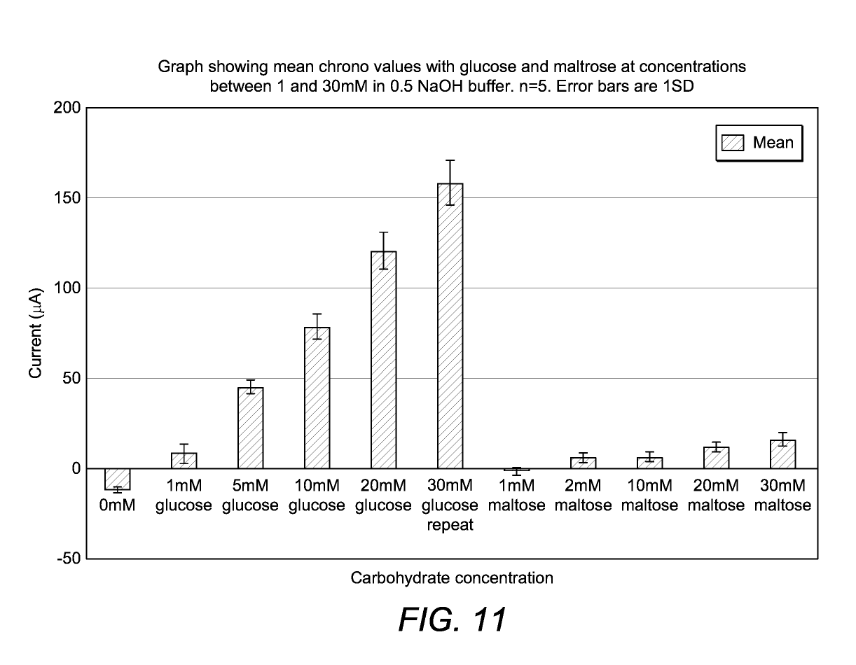

To demonstrate that ACuTEGA has adequate discrimination against maltose, calibration solutions for each sugar were prepared with concentrations between 1 mM and 30 mM. These were assayed by ACuTEGA under identical conditions, giving the results shown in FIG. 11.

The results in FIG. 11 indicate that at the high pH necessary for the operation of the ACuTEGA system, maltose at clinically relevant concentrations shows a much lower electrochemical response compared with glucose. With such a difference in response, one can be confident that an ACuTEGA glucose value in a patient with maltose as high as 30 mM (10 times the maximum reported clinical level) would at most be compromised by about 1 mM, which would not lead to a miss-reporting of blood glucose that would either wrongly deny glucose in a hypoglycaemic state, or wrongly identify a hyperglycaemic state, resulting in an overdose with insulin.

Creation of 1 μl Volume Capillary Chambers that Reliably Fill with Whole Blood from a Finger-Prick
Capillary chamber tops (self-adhesive) are standard units, and reels of suitable materials have been acquired on a research scale.
Approximately 1 μL void volumes are created, using hydrophilic capillaries.
Dried reagents are placed within the capillary chamber, which in turn are rehydrated when the test sample enters the capillary space.
   Reliable Deposition of Solid Sodium Hydroxide within the Chamber without Corroding the Ultra-Thin Copper Film and without Impeding Capillary Filling.
Pre-dosing the electrode chamber with correct volume and concentration of sodium hydroxide is critical to test operation.
The pH of the whole blood sample has to be raised above the ionisation point of glucose, higher than pH 13, before a measurement is taken (less than 5 seconds).
To achieve stability, hydroxide has to be present as a dry reagent presenting several issues:
Hydroxide contact with copper initiates a destructive process, so dry hydroxide cannot be stored in direct contact with the electrode surface.
Dry hydroxide has been used in submarines and spacecraft as a CO2 scrubber, in which the hydroxide reacts rapidly with carbon dioxide to form sodium carbonate. This reaction also occurs in ACuTEGA chambers when are open to the atmosphere. If the storage atmosphere is uncontrolled, over time, the pH of the dry reagent drops. If substantial conversion occurs, the blood pH is not raised high enough to ionise glucose.
Drying hydroxide pure from simple aqueous solution results in crystal formation that are too large to dissolve quickly (within seconds) to allow a measurement within the target timescale.
It was discovered that a carrier, or a “dispersing agent”, was required. A detergent, Proteric-JS, is used to allow the hydroxide to dry as far smaller crystals, thus increasing the surface area such that when the blood is applied the hydroxide can quickly dissolve.
   Dosing of Hydroxide without Loss of Potency in Storage (Through Reaction with Carbon Dioxide) with Instant, Uniform Alkalinisation of the Blood.
The surface area to volume (of the dry film) ratio is very large. For this reason, even though CO2 concentration in air is low (˜0.04%) enough is absorbed to force a pH drop. To overcome this, the pre-dosed sensors are packaged in the presence of molecular sieve. This material reduces the moisture content of the air within the packaging to almost complete dryness and simultaneously absorbs CO2.
The dried reagent is located on the capillary chamber surface, rather than directly on the copper electrode surface. Direct deposition of the hydroxide reagent onto copper is not effective due to the corrosive nature of the hydroxide.
In practice, the pre-dosed dry hydroxide almost instantly dissolves into the blood, raising the pH sufficiently to allow the copper oxidation chemistry to work.
   Performance of the Dried, Pre-Dosed System with 1 μL Chambers

The dried system operating with capillary chambers manufactured by hand on small-scale is vulnerable to some variation compared to electrodes of similar dimensions that are operated with wet reagents and larger sample volumes. Thus, the capillary chamber versions were subjected to rigorous performance testing to understand impact of manufacturing parameters on the resuspension of the dried reagents within the capillary chambers. The following data were obtained using fully dried and miniaturised devices.

### Linearity:

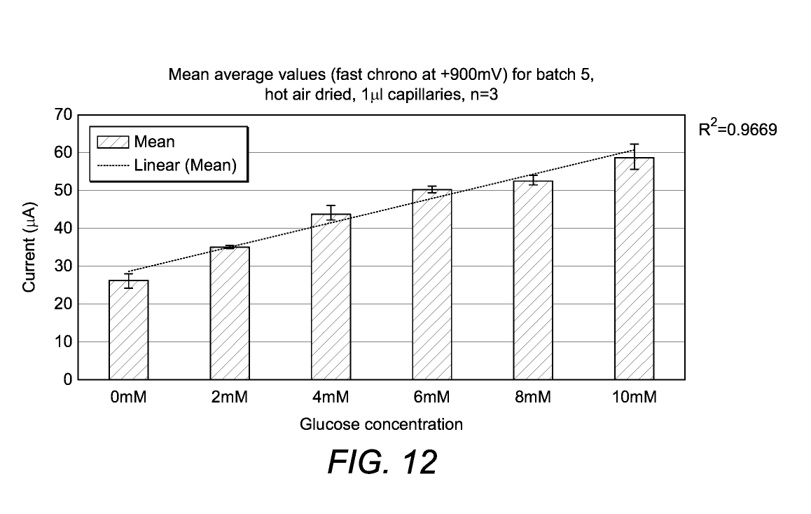

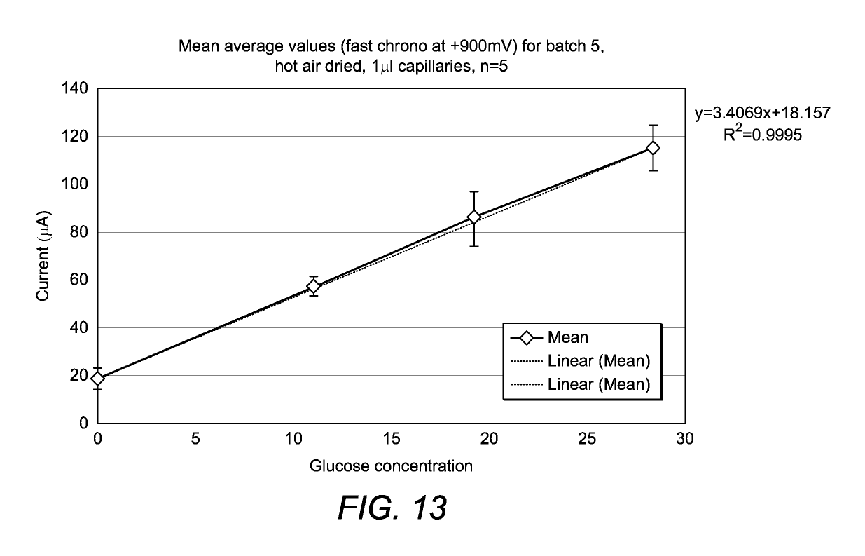

Excellent linearity is observed when testing either 0-10 mM (short range) and 0-30 mM (long range) in whole blood, as shown in FIGS. 12 and 13 respectively.

Correlation of ACuTEGA with a Reference Device:

The ACuTEGA device is used to measure glucose in blood during a non-fasting glucose tolerance test. A non-diabetic volunteer consumes a glucose containing drink. A finger-prick blood sample is tested by ACuTEGA, the YSI STAT Plus analyser, and a commercial self test blood glucose systems, the Bayer Contour XT.

Capillary blood is drawn via lancet puncture of a finger. A 1 μL drop of blood is applied to the ACuTEGA capillary chamber. Electrochemical measurements are made by the “fast chrono” method, as previously described. Another sample of blood from the same puncture is also measured by the YSI analyser and the Contour XT device. Blood glucose levels are measured every 30 minutes following consumption of the glucose containing drink over a 2 hour period using each device. The level of glucose within a first blood sample represents a baseline level; the level of glucose within a second blood sample will increase above the baseline; the level of glucose in a third and subsequent blood samples is similar to the baseline. Signals from each technology correspond to the expected glucose levels and the changes exhibited by the signals measured using the copper electrode are correlated to the changes in glucose levels determined using the classic technologies.

1.-10. (canceled)

11. A device for determining the glucose content of a sample comprising a sample analysis area wherein the sample analysis area comprises electrodes and pre-deposited reagent for alkalinisation of the sample.

12. The device of claim 11 wherein the electrodes comprise metals or conducting polymers.

13. The device of claim 11 wherein:
a. the electrodes comprise copper working electrode, a silver/silver chloride reference electrode and a platinum counter electrode
b. the working, counter and reference electrodes are all gold
c. the working and counter electrodes are gold and the reference electrode is silver/silver chloride
d. the electrodes comprise gold working electrode, a silver/silver chloride reference electrode and a platinum counter electrode
e. the working, counter and reference electrodes are all copper; or
f. the working and counter electrodes are copper and the reference electrode is silver/silver chloride.

14. The device of claim 11 wherein the (copper and platinum) electrodes comprise evaporated film electrodes.

15. The device of claim 11 wherein the reagent for alkalinisation of glucose comprises a strong base, optionally wherein the strong base comprises sodium hydroxide, potassium hydroxide, Barium hydroxide, ammonium, ammonium hydroxide or methylammonium.

16. The device of claim 11 wherein the reagent for alkalinisation of glucose further comprises a polyion, optionally wherein the polyion comprises EDTA and or polyethyleneimine.

17. The device of claim 11 wherein the reagent for alkalinisation for the sample further comprises a surfactant.

18. The device of claim 11 wherein the electrodes and reagent for alkalinisation of the sample are physically separate but fluidically connected.

19. The device of claim 11 where the electrodes are capable of electro-catalysis of ionised glucose.

20. The device of claim 13 wherein the electrodes comprise alternative electrode arrangements.

21. The device of claim 11 wherein glucose is determined electrochemically following ionisation and electrocatalysis of glucose.

22. The device of claim 11 wherein the glucose can be determined at more than one electrode potential.

23. A biosensor, comprising;
a base layer having disposed thereon at least one conductive track extending from a first end to a second end, wherein the conductive track comprises copper;
an assay zone at the first end of the base layer, comprising a reagent capable of increasing the pH of a sample applied to the assay zone;
a terminal at the second end of the base for connection of the at least one conductive track to a processor.

24. The biosensor of claim 23 further comprising a capillary chamber at the first end for receiving a sample of body fluid, wherein the capillary chamber is disposed over the assay zone such that a portion of the at least one conductive track is exposed within the capillary chamber.

25. The biosensor of claim 23, wherein the base layer has disposed thereon at least three conductive tracks, each conductive track being electrically insulated from the other, optionally wherein the at least three conductive tracks comprise copper and wherein a portion of the at least three conductive tracks is exposed within the capillary chamber, and further wherein the capillary chamber contains the pH altering reagent.

26. The biosensor of claim 23 wherein the pH altering reagent is disposed:
a. on an inner surface of the capillary chamber;
b. on the base layer, but not in contact with the at least three conductive tracks within the capillary chamber; and/or
c. within the capillary chamber.

27. The biosensor of claim 23 wherein the at least three conductive tracks define at least one measurement electrode, at least one reference electrode and at least one counter electrode, and wherein the measurement electrode, counter electrode and reference electrode are located within the capillary chamber in the assay zone.

28. A method, comprising:
ionizing glucose present in whole blood; and
electrochemically determining the presence of the ionized glucose in the whole blood, wherein ionizing the glucose comprises combining the whole blood with a dried reagent, optionally wherein the dried reagent is present in an amount sufficient to increase the pH of the whole blood by an amount sufficient to ionize the glucose.

29. (canceled)

30. The method of claim 28, wherein the electrochemically determining is performed in a chamber having a total volume of less than about 5 microliters.

31. The method of claim 28, wherein the electrochemically determining comprises electrochemically determining the ionized glucose via an electrochemical circuit comprising at least one copper electrode in contact with the whole blood.

32. The method of claim 28, wherein the method is performed in the absence of enzymes/mediators.

33. A test strip for determining the presence of glucose, comprising:
a capillary chamber defining a total volume of less than about 2.5 microliters;
at least one copper electrode in electrochemical communication with the capillary chamber; and
a dried reagent present in an amount sufficient to increase a pH of a whole blood sample introduced into the capillary chamber and filling the volume of the capillary chamber by an amount sufficient to ionize glucose present in the whole blood.

34. The test strip of claim 33 wherein the test strip comprises three copper electrodes configured as:
i) a working electrode at which measurement of glucose oxidation occurs;
ii) a counter electrode, which supplies or consumes electrons in response to the reaction at the working electrode; and
iii) a reference electrode, which acts to monitor and maintain the potential applied between the working electrode and counter electrode.

35. The test strip of claim 33 wherein the capillary chamber defines a volume of less than about 2 microlitres, less than about 1 microlitre or less than about 0.5 microlitres.

36. The test strip of claim 33 wherein the dried reagent is disposed on a surface of the capillary chamber not in direct contact with the one or more copper electrodes.

37. The test strip of claim 33 wherein the dried reagent comprises a base and a surfactant, optionally wherein the surfactant is polyvinyl alcohol and the base is sodium hydroxide.

38. A method of determining the quantity of glucose in a sample of blood obtained from a finger prick or alternate site using the test strip of claim 33, comprising;
removing the test strip from a storage compartment;
inserting the test strip into a meter and following the instructions presented on the display of the meter;
pricking a finger or alternate site to release a drop of blood;
contacting the drop of blood with the sample port on the test strip;
removing the test strip from the drop of blood when the meter indicates sufficient sample has been acquired on the test strip;
allowing the blood to react in the test strip for at least 1 second; and
displaying a blood glucose concentration on the display of the meter.

39. The method of claim 38, wherein the blood reacts in the test strip for at least 3, 5, 7 or 10 seconds before a glucose concentration is displayed.

40. The method of claim 38 wherein no more than 2.5, 1.5, 1 or 0.5 microlitres of blood has been acquired on the test strip.
